(19) United States
(12) Patent Application Publication
(10) Pub. No.: US 2020/0179575 A1
(43) Pub. Date: Jun. 11, 2020

(54) Liquid Collection Container For Negative-Pressure Therapy

(71) Applicant: KCI Licensing, Inc., San Antonio, TX (US)

(72) Inventors: Christopher Brian LOCKE, Bournemouth (GB); Richard Daniel John COULTHARD, Verwood (GB)

(21) Appl. No.: 16/631,785

(22) PCT Filed: Jul. 20, 2018
(86) PCT No.: PCT/US2018/043141

§ 371 Date: Jan. 16, 2020

Related U.S. Application Data
(60) Provisional application No. 62/539,436, filed on Jul. 31, 2017.

(51) Int. Cl. A61M 1/00

(52) U.S. Cl. CPC A61M 1/0001; A61M 1/0049; A61M 2205/3382; A61M 2205/7536; A61M 2205/273

(57) ABSTRACT

In an example is a system for treating a tissue site. The system may comprise a container having a liquid reservoir adapted to be fluidly coupled to the dressing. The container may comprise a liquid-degradable component configured to allow gas communication between the liquid reservoir and an external environment upon a liquid level within the liquid reservoir reaching a predetermined level. The liquid-degradable component may also be configured to restrict gas communication between the liquid reservoir and the external environment prior to the liquid level within the liquid reservoir reaching the predetermined level. The container may also comprise a hydrophobic filter configured to allow gas communication and to restrict liquid communication between the liquid reservoir and the external environment. The system may also comprise a negative-pressure source adapted to be fluidly coupled to the container.

### RELATED APPLICATIONS

The present invention claims the benefit, under 35 U.S.C. § 119(e), of the filing of U.S. Provisional Patent Application Ser. No. 62/539,436, filed Jul. 31, 2017. This provisional application is incorporated herein by reference for all purposes.

### TECHNICAL FIELD

The subject matter disclosed herein and recited in the appended claims relates generally to tissue treatment systems and more particularly, but without limitation, to apparatuses, systems, and methods for the treatment of a tissue site with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating tissue sites, particularly, wounds. Regardless of the etiology of a wound, whether trauma, surgery, or another cause, proper care of the wound is important to the outcome. Treatment of wounds or other tissue sites with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for a therapy including the provision of negative pressure are set forth in the appended claims. Illustrative embodiments are also provided to enable a person skilled in the art to make and use the claimed subject matter.

For example, in some embodiments is a system for treating a tissue site with reduced pressure. The system may comprise a dressing adapted to be placed adjacent to the tissue site. The system may also comprise a container having a liquid reservoir adapted to be fluidly coupled to the dressing. The container may comprise a liquid-degradable component configured to allow gas communication between the liquid reservoir and an external environment upon a liquid level within the liquid reservoir reaching a predetermined level. The liquid-degradable component may also be configured to restrict gas communication between the liquid reservoir and the external environment prior to the liquid level within the liquid reservoir reaching the predetermined level. The container may also comprise a hydrophobic filter configured to allow gas communication and to restrict liquid communication between the liquid reservoir and the external environment. The system may also comprise a negative-pressure source adapted to be fluidly coupled to the container.

Further, in some embodiments is a container for collecting liquid from a dressing in a negative-pressure treatment system. The container may comprise a liquid reservoir, a first port, and a liquid-degradable component. The liquid-degradable component may be configured to allow gas communication between the liquid reservoir and an external environment upon a liquid level within the liquid reservoir reaching a predetermined level. The liquid-degradable component may also be configured to restrict gas communication between the liquid reservoir and the external environment prior to the liquid level within the liquid reservoir reaching the predetermined level. The container may also comprise a hydrophobic filter configured to allow gas communication and to restrict liquid communication between the liquid reservoir and the external environment.

Further still, in some embodiments is a method for treating a tissue site with reduced pressure. The method may comprise disposing a dressing adjacent to the tissue site. The method may also comprise fluidly coupling a negative-pressure source to the dressing. The method may also comprise drawing fluid from the tissue site with the negative-pressure source. The method may also comprise collecting at least a portion of the fluid in a liquid reservoir of a container fluidly coupled between the dressing and the negative-pressure source. The container may comprise a liquid-degradable component configured to restrict gas communication between the liquid reservoir and an external environment prior to a liquid level within the liquid reservoir reaching a predetermined level and to allow gas communication between the liquid reservoir and the external environment upon the liquid level within the liquid reservoir reaching the predetermined, level. The container may also comprise a hydrophobic filter configured to allow gas communication and to restrict liquid communication between the liquid reservoir and the external environment.

In some embodiments, a container for collecting liquid from a dressing in a reduced pressure treatment system may be effective to cease the application of negative pressure to a dressing and/or to cease the withdrawal of fluids from the tissue site upon the liquid within a liquid reservoir of the container reaching a, predetermined level and a liquid-degradable component being degraded. Additionally or alternatively, the loss of negative pressure from the liquid reservoir upon the liquid within the liquid reservoir reaching the predetermined level and degradation of the liquid-degradable component, may provide an indication that the liquid within the liquid reservoir has reached the predetermined level. Additionally or alternatively, in some embodiments a container for collecting liquid from a dressing in a reduced pressure treatment system may ensure that the container is used only once. For example, upon the liquid-degradable component degrading and allowing pressure to be communicated between the liquid reservoir and the external environment, the container may be effectively unable to retain a negative pressure. For example, because the container will not retain a negative pressure applied thereto, the container may be unusable in any future therapies. For example, by being rendered unusable in any additional therapies, the container may reduce the risk that a previously-used, unsterile container might be reused.

Objectives, advantages, and illustrative modes of making and using the claimed subject matter may be understood best by reference to the accompanying drawings in conjunction with the following detailed description of illustrative embodiments.

### BRIEF DESCRIPTION OF THE DRAWINGS

FIG. 1 is a functional schematic of an embodiment of a therapy system for use in providing negative pressure therapy in accordance with this specification.

FIG. 2 is a cutaway view of an embodiment, a container for use in a therapy system.

FIG. 3 is a detailed view of a portion of the container of FIG. 2.

FIG. 4 is a cutaway view of an embodiment a container for use in a therapy system.

FIG. 5 is a detailed view of a portion of the, container of FIG. 4.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

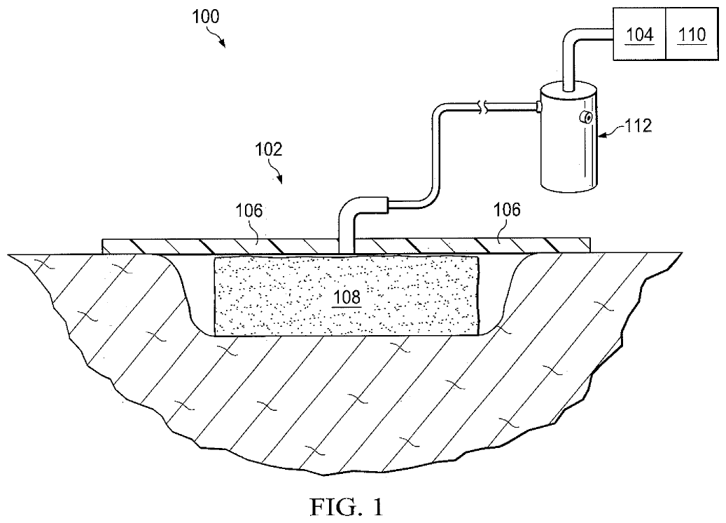

FIG. 1 is a simplified functional diagram of an example embodiment of a system 100 that can provide negative-pressure therapy to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The system 100 may include a negative-pressure supply, and may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be fluidly coupled to a negative-pressure source 104, as illustrated in FIG. 1. A dressing may include a cover, a tissue interface, or both in some embodiments. The dressing 102, for example, may include a cover 106 and a tissue interface 108. A regulator or a controller, such as a controller 110, may also be coupled to the negative-pressure source 104. In some embodiments, a dressing interface may facilitate coupling the negative-pressure source 104 to the dressing 102. For example, such a dressing interface may be the SENSAT.R.A.C.™ Dressing available from KCI of San Antonio, Tex.

Additionally, the system 100 may include sensors to measure operating parameters and provide feedback signals to the controller 110 indicative of the operating parameters. For example, the system 100 may include a pressure sensor, an electric sensor, or both, coupled to the controller 110. The pressure sensor may also be coupled or configured to be coupled to a distribution component and to the negative-pressure source 104.

Components may be fluidly coupled to each other to provide a path for transferring fluids (i.e., liquid and/or gas) between the components. For example, components may be fluidly coupled through a fluid conductor, such as a tube. A “fluid conductor,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. In general, components of the system 100 may be coupled directly or indirectly.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many eases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, open-cell foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. In one non-limiting example, the tissue interface 108 may be a reticulated polyurethane foam such as the foam employed in the V.A.C.® GRANUFOAM™ Dressing or the V.A.C.® VERAFLO™ Dressing, both available from KCI of San Antonio, Tex.

The tissue interface 108 may be either hydrophobic or hydrophilic. In an example in which the tissue interface 108 may be hydrophilic, the tissue interface 108 may also wick liquids away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as the foam employed in the V.A.C. WHITEFOAM™ Dressing available from KCI of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m² per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the system 100, such as the negative-pressure source 104. In some embodiments, for example, the controller 110 may be a microcontroller, which generally comprises an integrated circuit containing a processor core and a memory programmed to directly or indirectly control one or more operating parameters of the system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals.

Sensors, such as the pressure sensor or the electric sensor, are generally known in the art as any apparatus operable to detect or measure a physical phenomenon or property, and generally provide a signal indicative of the phenomenon or property that is detected or measured. For example, the pressure sensor and the electric sensor may be configured to measure one or more operating parameters of the system 100. In some embodiments, the pressure sensor may be a transducer configured to measure pressure in a pneumatic pathway and convert the measurement to a signal indicative of the pressure measured. In some embodiments, for example, the pressure sensor may be a piezoresistive strain gauge. The electric sensor may optionally measure operating parameters of the negative-pressure source 104, such as the voltage or current, in some embodiments. Preferably, the signals from the pressure sensor and the electric sensor are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

The system 100 may include a liquid container, such as a container 112, coupled to the dressing 102 and to the negative-pressure source 104. For example, in some embodiments a tube may mechanically and fluidly couple the dressing 102 to the container 112. The container 112 is representative of any suitable container, canister, pouch, or other storage component, which can be used to manage exudates and other liquids withdrawn from a tissue site. The container 112 may be configured to restrict gas communication between an interior space of the container and an external environment prior to a liquid level within the container reaching a predetermined level and to allow gas communication between the interior space and the external environment upon the fluid level within the container 112 reaching the predetermined level.

### Container

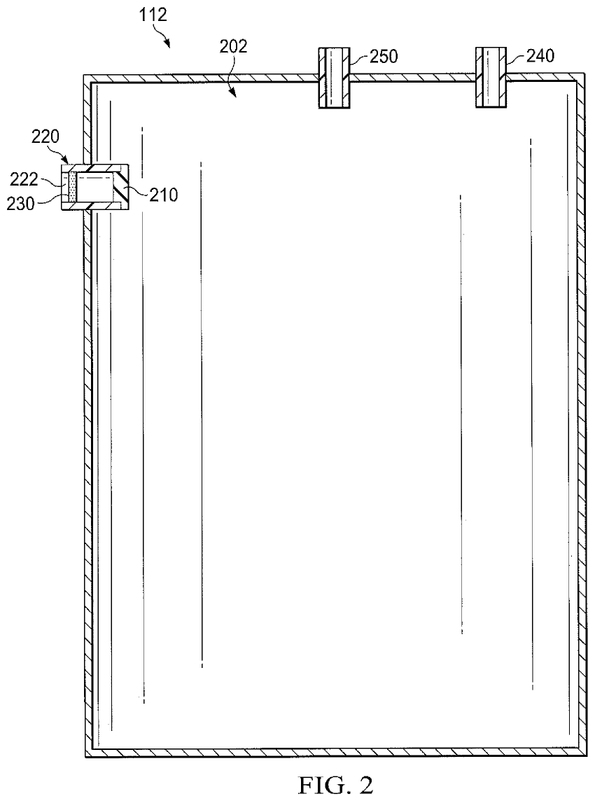

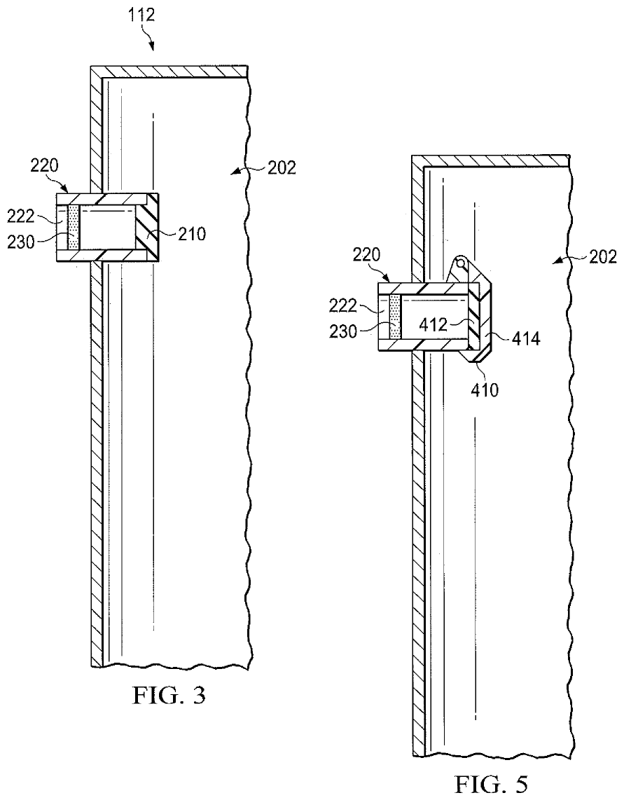

In many environments, a rigid container may be preferred or required for collecting, storing, and disposing of liquids. For example, FIGS. 2 and 3 illustrate an example embodiment of the container 112 which may be rigid and define a liquid reservoir 202 having a fixed internal volume. In some embodiments, the container 112 may include sidewalls, a base, and a top generally defining the liquid reservoir 202 having a fixed internal volume. For example, the liquid reservoir 202 may define an internal volume from about 0.5 L to about 2.5 L or, in some embodiments, from about 1.0 to about 1.5 L.

In various embodiments, the container 112 may have any suitable shape, design, and orientation. In some embodiments, for example, the container 112 may be described as generally conical, tapered, pyramidal, or cubic. Also, the container 112 may be described as having a cross-section in a horizontal plane that is circular, oval, square, rectangular, triangular, pentagonal, hexagonal, or any other suitable shape.

The liquid reservoir 202 may be generally adapted to be substantially fluid-tight, for example, such that a negative pressure applied to the liquid reservoir 202 of the container 112 may be retained with little dissipation of the negative pressure. In some embodiments, the container 112 may comprise a cover or lid. The engagement between the lid and the container 112 may include a suitable seal, examples of which include but are not limited to, an O-ring, a T-seal, a gasket, and a compression seal, as suitable.

In some embodiments, the container 112 may be configured to restrict gas communication between the liquid reservoir 202 and an external environment prior to a liquid level within the liquid reservoir reaching a predetermined level and to allow gas communication between the liquid reservoir 202 and the external environment upon the fluid level within the liquid reservoir 202 reaching the predetermined level. In various embodiments, the predetermined level at which the container 112 may be configured to allow gas communication between the liquid reservoir 202 and the external environment may be where a predetermined percentage of the liquid reservoir 202 contains liquid, for example, where the liquid reservoir 202 is about 90% full or substantially full. The container may define a first route of fluid communication between the liquid reservoir 202 and the external environment. For example, in some embodiments the container 112 includes a first port 220 defining a first flowpath 222 that provides the first route of fluid communication between the liquid reservoir 202 and the external environment. In various embodiments, the first port 220 may be disposed at any suitable position with respect to the container 112, for example, such that the predetermined level may be varied as desired. For example, the first port 220 may be disposed in a sidewall of the container 112 near the top of the container 112.

### Degradable Component

In some embodiments, the container 112 may comprise a fluid-degradable component configured to control fluid communication between the liquid reservoir 202 and the external environment. As used herein, “liquid-degradable” refers to a characteristic of a material or component to undergo a change in structure or composition upon contact with a liquid and resulting in the loss of structural integrity of the liquid-degradable component. For example, the fluid-degradable component may be configured to degrade upon sufficient contact with a liquid, such as blood, wound exudate, or aqueous liquids. In some embodiments, sufficient contact between the liquid-degradable component and a suitable liquid may cause the liquid-degradable component to be wholly or substantially eroded, dissolved, or disintegrated. For example, erosion, dissolution, or disintegration of at least a portion of the liquid-degradable component may cause the liquid-degradable component to be weakened and lose structural integrity as a result of other forces, such as a pressure-differential, applied to the liquid-degradable component. For example, in some embodiments the liquid-degradable component may include lines of weakness, for example, such that the liquid-degradable component may burst, collapse, or otherwise fail structurally upon erosion, dissolution, and/or disintegration of less than the entire liquid-degradable component.

In some embodiments, the liquid-degradable component may comprise or be formed from a suitable liquid-degradable material. Examples of suitable degradable materials for use in the liquid-degradable component include, but are not limited to, salts and water-soluble polymers such as polyvinyl alcohol (PVA), hydrogels, and cellulose derivatives such as carboxymethyl celluose (CMC). For example, in some embodiments the liquid-degradable material may be compressed, molded, milled, or otherwise formed into the liquid-degradable component. In some embodiments, the liquid-degradable material may further comprise one or more suitable additives. For example, the liquid-degradable material may include a dye, for example, such that degradation of the liquid-degradable component upon contact with a liquid may cause the dye to be released into the liquid.

In some embodiments, the liquid-degradable component may be configured such that a desired duration of contact between the liquid-degradable component and the liquid is effective to cause the liquid-degradable component to be eroded, dissolved, or disintegrated sufficiently to lose structural integrity. For example, the duration of contact between the liquid-degradable component and the liquid effective to yield the loss of structural integrity may be from about instantaneous to about 5 minutes, or from about 10 seconds to about 3 minutes, or from about 30 seconds to about 2 minutes. In some embodiments, the liquid-degradable component may comprise a degradation modifier configured to modify the duration of contact between the liquid-degradable component and the liquid effective to yield the loss of structural integrity. In some embodiments, the liquid-degradable component may comprise a hydrophilic coating or a hydrophobic coating.

In some embodiments, the container 112 may comprise a liquid-degradable component configured as a stopple 210. The stopple 210 may be configured to control fluid communication via the first route of fluid communication and, thereby, control fluid communication between the liquid reservoir 202 and the external environment, for example, via the first flowpath 222. For example, the stopple 210 may be configured to restrict gas communication via the first port 220 prior to being degraded and to allow gas communication via the first port 220 upon being sufficiently degraded.

In some embodiments, the stopple 210 may be configured to cover, block, engage, or otherwise interact with the first port 220. For example, in some embodiments the stopple 210 may comprise a plug, an insert, a cap, a disc, an insert, a cover or other suitable configuration. The stopple 210 may be engaged with or positioned within the first port 220, for example, such that when engaged with or positioned within the first port 220, the stopple 210 does not allow pressure to be communicated via the first port 220. For example, in various embodiments, the stopple 210 may engage the first port 220 via a suitable interface, for example, a threaded interface or a frictional interface. Additionally or alternatively, in some embodiments the stopple 210 may be trapped within and/or with respect to the first flowpath 222, for example, by a suitably-configured retaining member.

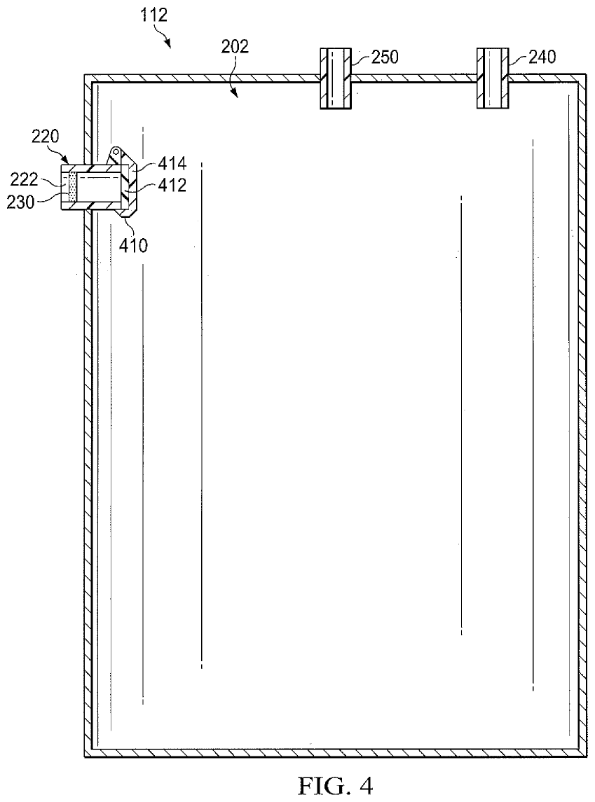

Additionally or alternatively, in some embodiments the liquid-degradable component may be configured to retain another member relative to the first port 220. For example, in some embodiments the container 112 may further comprise a cap or other cover, which may be configured to prevent pressure communication via the first port 220 when positioned relative to the first port 220. The liquid-degradable component may be configured to retain the cap or cover relative to the first port 220. For example, in the embodiment of FIGS. 4 and 5, the liquid-degradable component may comprise a retainer 410, such as a pin, a retaining clip, an anchor, or the like. For example, in the embodiment of FIGS. 4 and 5 the container 112 may further comprise a cap 412 disposed on an arm 414. The arm 414 may be configured to render the cap 412 rotatable between a first position in which the cap 412 restricts fluid communication via the first port 220 and a second position in which the cover does not restrict fluid communication via the first port 220. In some embodiments, the arm 414 may be biased, for example, toward the second position, via a biasing member such as a spring or via a weight and/or cantilever. In some embodiments, the retainer 410 may be configured to retain the cap 412, the arm 414, or both, such that the cap 412 remains in the first position prior to degradation of the retainer 410,

Additionally or alternatively, in some embodiments the liquid-degradable component may be configured such that degradation of the liquid-degradable component causes indicia of the degradation to be presented to a user. In various embodiments, degradation of the liquid-degradable component may cause auditory indicia and/or visual indicia of the degradation to be presented to a user. For example, in some embodiments, the first port 220 may be configured to create an audible signal. For example, the first port 220 may be configured as an aerophone (e.g., a whistle), such that the movement of air through the first flowpath 222 upon degradation of the liquid-degradable component produces a “whistling” sound. Additionally or alternatively, in some embodiments the degradation of the liquid-degradable component may be configured to actuate a switch, for example, via movement of the arm 414 between the first position and the second position, so as to cause a signal to be communicated. In some embodiments, the signal may be effective to cause an alarm to be triggered, to cause the negative-pressure source 104 cease operation, or combinations thereof. Additionally or alternatively, in some embodiments, degradation of the liquid-degradable component may release a dye into a liquid within the liquid reservoir 202. For example, the liquid within the liquid reservoir 202 may undergo a visual change such as a color change upon degradation of the liquid-degradable component.

### Hydrophobic Filter

In some embodiments, the container 112 may further comprise a hydrophobic filter 230 configured to control liquid communication between the liquid reservoir 202 and the external environment. For example, the hydrophobic filter may be configured to control liquid communication via the first flowpath 222.

In some embodiments, the hydrophobic filter 230 may be generally configured to restrict liquid communication and to allow gas communication. For example, the hydrophobic filter 230 may comprise a material that is generally liquid impermeable and vapor permeable. The hydrophobic filter 230 may be, for example, a porous, sintered polymer. As an example, the hydrophobic filter 230 may comprise a material manufactured under the designation MMT-314 by W.L. Gore & Associates, Inc. of Newark, Del., or similar materials.

In some embodiments, the hydrophobic filter 230 may be configured to interact with the first port 220, for example, so as to substantially preclude liquid from passing through the first port 220 via the first flowpath 222. For example, in some embodiments the hydrophobic filter 230 may comprise a cylinder or other insert sized to fit the dimensions of the first flowpath 222. In some embodiments, the hydrophobic filter 230 may be in the form of a membrane or layer. The hydrophobic filter 230 may be disposed within and/or over the first port 220. For example, in various embodiments, the hydrophobic filter 230 may be held in place with respect to the first port 220 via a mechanical interface such as a threaded interface or a frictional interface or by an adhesive.

### Additional Fluid Ports

In some embodiments, the container 112 may also be configured to provide one or more suitable routes of fluid communication to and/or from one or more other components of the system 100. For example, in some embodiments, the container 112 may further include a suitable number of connection ports. In various embodiments, a connection port may include a suitable fitting or coupler, for example, to provide for connection to a fluid conduit. Examples of such fitting and couplers may include, but are not limited to, push-to-connect fittings, compression fittings, barb fittings, and the like. For example, in the embodiment of FIG. 2, the container 112 may include a second port 240 which may be configured to provide fluid communication between the liquid reservoir 202 and the negative-pressure source 104. Also for example, in the embodiment of FIG. 2, the container 112 may include a third port 250 which may be configured to provide a route of fluid communication between the dressing 102 and the liquid reservoir 202. In some embodiments, second port 240, the third port 250, or both may be disposed at a suitable position with respect to the container 112. The second port 240 and/or the third port 250 may be disposed at a height above the height of the first port 220, for example, such that the second port 240 and/or the third port 250 may remain unsubmerged upon a liquid reaching the first port 220.

### Methods

The container 112 may be employed in the context of a negative-pressure therapy, for example, to collect wound liquids, such as blood, water, and wound exudate, removed from a tissue site. In operation, the container 112 may be effective to retain a negative pressure applied to the liquid reservoir 202, for example, via the operation of the negative-pressure source 104, prior to the liquids within the liquid reservoir 202 reaching the predetermined level. The container 112 may also be effective to cause the negative pressure retained within the liquid reservoir 202 to be released or dissipated upon the liquids within the liquid reservoir 202 reaching the predetermined level. Also, in operation, the container 112 may be effective to provide an indication when the liquid level within the container 112 reaches the predetermined level, for example, dependent upon the placement of the liquid-degradable component. In various embodiments, the predetermined level may be any desired level or volume of liquid within the liquid reservoir. For example, in some embodiments, the predetermined level may be where the liquid reservoir is full or substantially full of liquid.

For example, in a therapy method, the tissue interface 108 may be placed within, over, on, or otherwise proximate to a tissue site. The cover 106 may be placed over the tissue interface 108 and sealed to an attachment surface near the tissue site. For example, the cover 106 may be sealed to undamaged epidermis peripheral to a tissue site. Thus, the dressing 102 can provide a sealed therapeutic environment proximate to a tissue site, substantially isolated from the external environment. The negative-pressure source 104 may supply negative pressure to reduce the pressure at the dressing 102.

In some embodiments, the application of negative pressure to the tissue site may be effective to withdraw or remove wound liquids from the tissue site. As the wound liquids are withdrawn from the tissue site, the liquids may be collected within the liquid reservoir 202 of the container 112. As the liquids within the liquid reservoir 202 collect, the liquid level within the liquid reservoir 202 may continue to rise and, as the liquid level rises, the liquids within the liquid reservoir 202 may come into contact with the liquid-degradable component, for example, about when the liquid reaches the predetermined level.

In some embodiments, contact between the liquid within the liquid reservoir 202 and the liquid-degradable component may cause the liquid-degradable component to begin to erode, dissolve, disintegrate, or otherwise degrade. Upon the liquid-degradable component degrading and losing structural integrity, gas communication between the liquid reservoir 202 and the external environment may be allowed, such as via the first flowpath 222. In some embodiments, such as the embodiment of FIGS. 2 and 3, sufficient contact between the liquid within the liquid reservoir 202 and the liquid-degradable material may degrade so that the stopple 210 loses structural integrity and fails to prevent gas communication via the first flowpath 222. Additionally or alternatively, in some embodiments, such as the embodiment of FIGS. 4 and 5, upon sufficient contact between the liquid within the liquid reservoir 202 and the liquid-degradable material the retainer 410 may be degraded such that the liquid-degradable component loses structural integrity and allows the arm 414 to rotate from the first position to the second position, for example, so that the cap 412 does not restrict fluid communication via the first port 220.

In some embodiments, while the container 112 may be configured to allow gas communication with the external environment via the first flowpath 222 upon degradation of the liquid-degradable component, the container 112 may be configured to continue to restrict liquid communication through the first flowpath 222. For example, in some embodiments, the hydrophobic filter 230 may allow gas flow through the first flowpath 222 while restricting liquid flow through the first flowpath 222. As such, the container 112 may allow gas communication while preventing any loss of the liquid within the liquid reservoir 202, for example.

In some embodiments, the first port 220 may be configured to allow dissipation of the negative pressure retained within the liquid reservoir 202 of the container 112 upon degradation of the stopple 210. For example, the first port 220 may be configured to allow gas communication via the first flowpath 222 at a rate greater than the rate at which the negative-pressure source 104 is effective to reduce the pressure within the liquid reservoir 202. Additionally, in some embodiments the container 112 may further comprise one or more similarly-configured liquid-degradable components, for example, to allow pressure to be dissipated more quickly or as a redundant or failsafe.

In some embodiments, the loss of negative pressure from the liquid reservoir 202 via the first flowpath 222 may be effective to cease the application of negative pressure to the dressing 102 and/or to cease the withdrawal of liquid from the tissue site. For example, the absence of a substantial pressure differential between the liquid reservoir 202 and the tissue site may cease withdrawal of liquids from the tissue site.

Additionally, in some embodiments, the loss of negative pressure from the liquid reservoir 202 upon degradation of the liquid-degradable component may provide an indication that the liquids within the liquid reservoir 202 has reached the predetermined level. For example, in some embodiments, the controller 110 may be configured to detect the presence of a leak from or within the system 100, such as from or within the container 112 or another component in fluid communication therewith. For example, prior to degradation of the liquid-degradable component, flow into the container 112, such as an influx of pressure or liquid, may be limited to liquids flowing from the dressing 102. In some embodiments, the controller 110 may be configured to recognize a series of conditions as indicative of a leak within the system 100, such as a pressure or fluid leak from or within the container 112 or another component in fluid communication with the container 112. For example, the controller 110 may employ, for example, an algorithm effective to relate the operation of the negative-pressure source 104, the duration over which the negative-pressure source 104 is operated, the pressure within the internal volume of the container 112, the internal volume of the container 112, or combinations, to recognize conditions indicative of a leak. In some embodiments, when the controller 110 determines the presence of a leak, the controller 110 may be further configured to provide an alarm, such as an indicator light, an icon on a touch screen, an audible alarm, a tactile or vibratory alarm, or a message transmitted remotely. Additionally or alternatively, if the controller 110 determines the presence of a leak, the controller 110 may be configured to discontinue operation of the negative-pressure source 104, for example, to shut-down the negative-pressure source 104. In such embodiments, the loss of negative pressure from the liquid reservoir 202 upon degradation of the liquid-degradable component may cause the controller 110 to indicate the presence of a leak and to provide an alarm and/or discontinue operation of the negative-pressure source 104.

Additionally or alternatively, in some embodiments, degradation of the liquid-degradable component may cause indicia of the degradation to be presented to a user, which may indicate liquid in the liquid reservoir 202 may have reached the predetermined level. For example, the indicia may include at least one of auditory indicia and visual indicia that may be perceived by a user, indicating that the liquid reservoir 202 is full or substantially full.

### Advantages

In various embodiments, a therapy system like system 100 or components thereof, such as the container 112, may be advantageously employed in the provision of negative pressure therapy to a patient. For example, the container 112 may be effective to cease the application of negative pressure to the dressing 102 and/or to cease the withdrawal of liquids from the tissue site upon the liquid within the liquid reservoir 202 reaching the predetermined level and degradation of the liquid-degradable component. Additionally or alternatively, the loss of negative pressure from the liquid reservoir 202 upon the liquid within the liquid reservoir 202 reaching the predetermined level and degradation of the liquid-degradable component may provide an indication that the liquid within the liquid reservoir 202 has reached the predetermined level.

Additionally, in various embodiments the container 112 may be effective to ensure that the container 112 is used only once. For example, upon the liquid-degradable component degrading and allowing pressure to be communicated via the first flowpath 222, the container 112 may be unable to retain a negative pressure effectively, which can render the container 112 unusable for subsequent treatment and reduce the risk of cross-contamination.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and the like. Typically, the term “about” refers to deviations that are greater or lesser than a stated value or range of values by 1/10 of the stated value(s), for example, ±10%. For instance, a concentration value of “about 30%” refers to a concentration between 27% and 33%. Each value or range of values preceded by the term “about” is also intended to encompass the embodiment of the stated absolute value or range of values. Whether or not modified by the term “about,” quantitative values recited in the claims include equivalents to the recited values, for example, deviations from the numerical quantity, but would be recognized as equivalent by a person skilled in the art.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102 may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A system for treating a tissue site with reduced pressure, the system comprising:
a dressing adapted to be placed adjacent to the tissue site;
a container having a liquid reservoir adapted to be fluidly coupled to the dressing, the container comprising a liquid-degradable component configured to allow gas communication between the liquid reservoir and an external environment upon a liquid level within the liquid reservoir reaching a predetermined level; and
a negative-pressure source adapted to be fluidly coupled to the liquid reservoir.

2. The system of claim 1, wherein the container comprises:
a first port configured to provide gas communication between the liquid reservoir and the external environment upon the liquid level within the liquid reservoir reaching the predetermined level;
a second port adapted to provide fluid communication between the liquid reservoir and the negative-pressure source; and
a third port adapted to provide a route of fluid communication between the dressing and the liquid reservoir.

3. The system of claim 2, wherein the liquid-degradable component is configured to restrict gas communication via the first port between the liquid reservoir and the external environment prior to the liquid level within the liquid reservoir reaching the predetermined level.

4. The system of one of claims 2-3, wherein the first port is configured to restrict liquid communication between the liquid reservoir and the external environment.

5. The system of one of claims 2-4, wherein the container comprises a hydrophobic filter configured to the control fluid communication via the first port.

6. The system of claim 5, wherein the hydrophobic filter is configured to allow gas communication and to restrict liquid communication.

7. The system of one of claims 2-6, wherein the first port is configured to engage the liquid-degradable component.

8. The system of claim 7, wherein the liquid degradable component comprises a plug.

9. The system of one of claims 7-8, wherein the liquid-degradable component comprises a salt or a water-soluble polymer.

10. The system of one of claims 1-9, wherein the gas communication between the liquid reservoir and the external environment upon the liquid level within the liquid reservoir reaching the predetermined level is effective to yield a loss of negative pressure within the container.

11. The system of claim 10, wherein the negative-pressure source is configured to detect the loss of negative pressure within the container.

12. The system of claim 11, wherein the negative-pressure source is configured to cease operation upon detection of the loss of negative within the container.

13. A container for collecting liquid from a dressing in a reduced pressure treatment system, the container comprising:
a liquid reservoir; and
a first port configured to engage a liquid-degradable component to restrict gas communication between the liquid reservoir and an external environment.

14. The container of claim 13, further comprising:
a second port adapted to provide fluid communication between the liquid reservoir and a negative-pressure source; and
a third port adapted to provide a route of fluid communication between the dressing and the liquid reservoir.

15. The container of one of claims 13-14, wherein the liquid-degradable component comprises a plug.

16. The container of one of claims 13-15, wherein the liquid-degradable component comprises a salt or a water-soluble polymer.

17. The container of one of claims 13-16, wherein the liquid-degradable component is configured to provide gas communication via the first port between the liquid reservoir and the external environment upon a liquid level within the liquid reservoir reaching a predetermined level.

18. The container of claim 17, wherein the liquid-degradable component is configured to restrict gas communication via the first port between the liquid reservoir and the external environment prior to the liquid level within the liquid reservoir reaching the predetermined level.

19. The container of one of claims 13-18, wherein the first port is configured to restrict liquid communication between the liquid reservoir and the external environment.

20. The container of one of claims 13-19, wherein the container comprises a hydrophobic filter configured to the control fluid communication via the first port.

21. The container of claim 20, wherein the hydrophobic filter is configured to allow gas communication and to restrict liquid communication.

22. The container of one of claims 13-21, wherein the gas communication between the liquid reservoir and the external environment upon the liquid level within the liquid reservoir reaching the predetermined level is effective to yield a loss of negative pressure within the container

23. A method for treating a tissue site with reduced pressure, the method comprising:
disposing a dressing adjacent to the tissue site;
fluidly coupling a negative-pressure source to the dressing;
drawing fluid from the tissue site with the negative-pressure source;
collecting at least a portion of the fluid in a liquid reservoir of a container fluidly coupled between the dressing and the negative-pressure source, wherein the container comprises a liquid-degradable component configured to restrict gas communication between the liquid reservoir and an external environment prior to a liquid level within the liquid reservoir reaching a predetermined level and allows gas communication between the liquid reservoir and the external environment upon the liquid level within the liquid reservoir reaching the predetermined level.

24. The method of claim 23, wherein the gas communication between the liquid reservoir and the external environment upon the liquid level within the liquid reservoir reaching the predetermined level is via a port.

25. The method of claim 24, wherein the port restricts liquid communication between the liquid reservoir and the external environment.

26. The method of one of claims 23-25, wherein the gas communication between the liquid reservoir and the external environment upon the liquid level within the liquid reservoir reaching the predetermined level is via a hydrophobic filter.

27. The method of claim 26, wherein the hydrophobic filter is configured to allow gas communication and to restrict liquid communication.

27. The method of one of claims 23-27, wherein upon the liquid level within the liquid reservoir reaching the predetermined level the liquid-degradable component is degraded to allow the gas communication between the liquid reservoir and the external environment.

28. The method of claim 27, wherein the liquid-degradable component comprises a plug.

29. The method of one of claims 27-28, wherein the liquid-degradable component comprises a salt or a water-soluble polymer.

30. The method of one of claims 23-29, wherein the gas communication between the liquid reservoir and the external environment upon the liquid level within the liquid reservoir reaching the predetermined level yields a loss of negative pressure within the container.

31. The method of claim 30, further comprising detecting the loss of negative pressure within the container via the negative-pressure source.

32. The method of claim 31, further comprising ceasing operation of the negative-pressure source upon detecting the loss of negative within the container.

33. A container for the collection of a wound liquid in a negative pressure therapy, as substantially disclosed herein.

34. A method comprising providing a reduced pressure to a tissue site via the container of claim 33.

35. A system comprising the container of claim 33 and a negative-pressure source.

36. A method comprising providing a reduced pressure to a tissue site via the system of claim 35.
